(19) United States
(12) Patent Application Publication
(10) Pub. No.: US 2018/0144819 A1
(43) Pub. Date: May 24, 2018

(54) USER DEVICE, SERVER, AND SYSTEM HAVING FUNCTION OF MANAGING BODY FAT OR GLUCOSE, AND METHOD FOR MANAGING BODY FAT OR GLUCOSE USING SAME

(71) Applicants: MASSCON CO., LTD., Seoul (KR); TEMPUS INC., Seoul (KR)

(72) Inventors: Yong Eui Lee, Gyeonggi-do (KR); Baek Kyu Shin, Gyeonggi-do (KR)

(21) Appl. No.: 15/573,473

(22) PCT Filed: May 12, 2016
(86) PCT No.: PCT/KR2016/004985

§ 371 Date: Nov. 12, 2017

(30) Foreign Application Priority Data
May 12, 2015 (KR) 10-2015-0066351

(51) Int. Cl. G16H 20/30; A61B 5/00; A61B 5/145; G16H 20/60; H04M 1/725

(52) U.S. Cl. CPC G16H 20/30; A61B 5/4872; H04M 1/72544; G16H 20/60; A61B 5/14532

(57) ABSTRACT

The present invention relates to a method of managing body fat or body sugar using a user device comprises transmitting a body fat or body sugar data to an external server, receiving application execution information according to the body fat or body sugar data from the external server, executing a body fat or body sugar related application based on the application execution information, and outputting an execution result of the body fat or body sugar related application, the application execution information is based on a result of levelizing the body fat or body sugar data.

### TECHNICAL FIELD

The present invention relates to manage a body fat or body sugar, more particularly, to a user device, a server and a system that be effectively able to manage body fat through a body fat or body sugar related applications.

### BACKGROUND ART

Modern peoples have interested in obesity and diet, as a living standard has increased in recent years. Especially, abdominal obesity induces adult diseases such as diabetes, hypertension and hyperlipidemia due to an increase of visceral fat, and there is growing concerns about an appearance due to an increase of belly fat.

As a result, the modern peoples are concentrating on dieting to reduce a body fat which is the main measure of obesity, and a variety of applications that encourage users to exercise or manage the user's exercise or chow intake among smartphone applications, are provided.

However, according to the related art, it is not possible to provide users with various feedbacks indicating results of measurement of a body fat or body sugar as a result of executing the application.

Further, according to the related art, the application cannot be executed differently according to a measurement result of the body fat or body sugar.

### DISCLOSURE OF THE INVENTION

### Technical Problem

It is an object of the present invention to provide a user device capable of effectively transmitting information related to a body fat to a user and actively managing and motivating the body fat.

It is another object of the present invention to realize an application that provides game character information or game information differentiated according to a measurement result of a body fat or body sugar.

### Technical Solution

According to another aspect of the present invention, there is provided a user device having a function of managing body fat or body sugar, the user device comprising: a storage unit for storing a body fat or body sugar related applications; a communication unit for transmitting body fat or body sugar data to an external server and receiving application execution information according to the body fat or body sugar data from the external server; a control unit for executing the body fat or body sugar related application based on the application execution information; and an output unit for outputting a result of execution of the body fat or body sugar related application.

Wherein the control unit controls operations of at least one of the storage unit, the communication unit, and the output unit, and the application execution information is based on a result of levelizing the body fat or body sugar data.

According to another aspect of the present invention, there is provided a server having a function of managing body fat or body sugar, the server comprising: a communication unit for receiving a body fat or body sugar data from a user device and transmitting application execution information corresponding to the body fat or body sugar data to the user device; a controller for analyzing the body fat or body sugar data, determining the application execution information according to the analysis result, and controlling the user device to execute a body fat or body sugar related application according to the determined application execution information; and a storage unit for storing at least one of the body fat or body sugar data and the application execution information. Wherein the control unit controls operations of at least one of the communication unit and the storage unit, and wherein the application execution information is based on a result of levelizing the body fat or body sugar data.

According to another aspect of the present invention, there is provided a system having a function of managing body fat or body sugar, the system comprising: a user device for transmitting a body fat or body sugar data; and a server for analyzing the body fat or body sugar data received from the user device and determining application execution information according to the analysis result. Wherein the user device executes a body fat or body sugar-related application corresponding to the application execution information received from the server, and the application execution information is based on a result of levelizing the body fat or body sugar data.

According to an aspect of the present invention, there is provided a method of managing body fat or body sugar using a user device, the method comprising: transmitting a body fat or body sugar data to an external server; receiving application execution information according to the body fat or body sugar data from the external server; executing a body fat or body sugar related application based on the application execution information; and outputting an execution result of the body fat or body sugar related application. The application execution information is based on a result of levelizing the body fat or body sugar data.

According to another aspect of the present invention, there is provided a method of managing body fat or body sugar using a server, the method comprising: receiving a body fat or body sugar data from a user device; analyzing the body fat or body sugar data and determining application execution information based on the analysis result; transmitting the determined application execution information to the user device; and controlling the user device to execute a body fat or body sugar related application in accordance with the determined application execution information. The application execution information is based on a result of levelizing the body fat or body sugar data.

According to another aspect of the present invention, there is provided a method of managing body fat or body sugar using a system, the method comprising: transmitting, by a user device, a body fat or body sugar data to a server; analyzing, by the server, the body fat or body sugar data received from the user device and determining application execution information according to the analysis result; transmitting, by the server, the determined application execution information to the user device; and executing, by the user device, a body fat or body sugar related application corresponding to the application execution information received from the server. The application execution information is based on a result of levelizing the body fat or body sugar data.

### Advantageous Effects

According to the embodiment of the present invention, the current body fat information, a change history of the body fat, a target body fat information, and the like are provided as various feedbacks according to a measurement result of a body fat or body sugar, thereby providing a body fat related information which are suitable to the user.

In addition, according to the embodiment of the present invention, it is possible to induce the user to reduce the body fat by providing differentiated character information or game information according to the measurement result of body fat or body sugar.

In addition, according to the embodiment of the present invention, various images or levels reflecting measurement results of body fat or body sugar are displayed through body fat or body sugar-related applications, thereby providing fun and interest in reducing body fat.

### BRIEF DESCRIPTION OF THE DRAWINGS

FIG. 1 illustrates a block diagram of a system having a body fat or body sugar management function according to an embodiment of the present invention.

FIGS. 2A and 2B illustrate a block diagram of a user device having a function managing a body fat or body sugar according to an embodiment of the present invention.

FIG. 3 illustrates a block diagram of a server having a function managing a body fat or body sugar according to an embodiment of the present invention.

FIG. 4 is a flowchart for illustrating a method of managing a body fat or body sugar according to an embodiment of the present invention.

FIGS. 5A and 5B illustrate a screen for a user information registration and a screen for a body fat or body sugar measurement according to an embodiment of the present invention, respectively.

FIGS. 6A to 6C illustrate a screen for displaying measurement results of body fat or body sugar according to an embodiment of the present invention.

FIGS. 7A and 7B illustrate a screen for displaying game character information and game information differentiated according to measurement results of a body fat or body sugar according to the embodiment of the present invention, respectively.

### MODE FOR CARRYING OUT THE INVENTION

The present invention will now be described more fully with reference to the accompanying drawings, in which exemplary embodiments of the invention are shown.

The invention may, however, be embodied in many different forms and should not be construed as being limited to the embodiments set forth herein; rather these embodiments are provided so that this disclosure will be thorough and complete, and will fully convey the concept of the invention to one of ordinary skill in the art. Meanwhile, the terminology used herein is for the purpose of describing particular embodiments only and is not intended to be limiting of exemplary embodiments.

Also, thickness or sizes of layers in the drawings are exaggerated for convenience of explanation and clarity, and the same reference numerals denote the same elements in the drawings. As used herein, the term “and/or” includes any and all combinations of one or more of the associated listed items.

The terminology used herein is for the purpose of describing particular embodiments only and is not intended to be limiting of exemplary embodiments. As used herein, the singular forms “a,” “an” and “the” are intended to include the plural forms as well, unless the context clearly indicates otherwise. It will be further understood that the terms “comprises” and/or “comprising” used herein specify the presence of stated features, integers, steps, operations, members, components, and/or groups thereof, but do not preclude the presence or addition of one or more other features, integers, steps, operations, members, components, and/or groups thereof.

In addition, a term such as a “unit”, a “module”, a “block” or like, when used in the specification, represents a unit that processes at least one function or operation, and the unit or the like may be implemented by hardware or software or a combination of hardware and software.

A user device 110 referred to herein may communicate with an external device (e.g., a body fat or body sugar measuring device or an external server) and may download and execute a predetermined application (e.g., a body fat or body sugar related application), and may include, for example, a smart phone, a smart TV, a tablet PC, a notebook, a desktop, and the like.

A body fat or body sugar related application referred to herein may include an application capable of providing various information or games utilizing the user's body fat, for example, information indicating a level of the user's body fat or a game differentiated by the level of the user's body fat. The body fat or body sugar related application may be installed in the user device, or downloaded and installed from an application provider server. In the latter case, downloading and installation may be performed upon input of a command signal from the user, or upon initial connection with a body fat or body sugar measuring device.

The body fat or body sugar measuring device may be a known device capable of acquiring a body fat information from a body of the user, and may be any device capable of transmitting a body fat information through an electrical signal. A diabetic healing application interworked with a game that can be processing differentially depending on an additional or alternative level of body sugar besides a level of body fat may be provided.

FIG. 1 illustrates a block diagram of a system 100 having a body fat or body sugar management function according to an embodiment of the present invention present invention.

Referring to FIG. 1, the system 100 according to the present invention may include a user device 110, an external server 120, and a body fat or body sugar measuring device 130. The user device 110 can acquire a body fat or body sugar data and transmit the body fat or body sugar data to the external server 120.

In one embodiment, the user device 110 may perform acquisition and transmission of body fat or body sugar data by executing a body fat or body sugar related application. In this case, the body fat or body sugar-related application may be executed by receiving an execution command from the user, or may be automatically executed upon detection of connection with the body fat or body sugar measuring device 130. A connection between the user device 110 and the body fat or body sugar measuring device 130 may be any interface capable of transmitting an electrical signal and may be a connection using a local wired/wireless communication network, a remote wired/wireless communication network, an internet network, a mobile communication network, bluetooth, VPN (virtual private network), pairing between devices, tethering including USB tethering, or Wi-Fi hotspot. An electrical signal of the body fat or body sugar measuring device 130 may be conveyed through a microphone module, which is an input device of the user device 110. However, the present invention is not limited thereto. The body fat or body sugar measuring device 130 may be a known body fat or body sugar measuring device. The user device 110 may be connected to the body fat measurement device or the body sugar measuring device over wire or wireless. The user device 110 can receive and levelize an information about a value of body fat or body sugar measured by the body fat or body sugar measuring device 130, to execute a corresponding management application. The management application may be a game application, which will be described in detail later.

A communication network between the user device 110 and the external server 120 may include a local wired/wireless communication network, a remote wired/wireless communication network, an internet network, a mobile communication network, a bluetooth, a virtual private network (VPN), pairing between devices, tethering including USB tethering or Wi-Fi hotspots. Also, the communication network may include relay devices such as hubs, gateways, and routers, as is well known in the art.

For example, a body fat or body sugar data may be detected using a body fat or body sugar sensor mounted on the user device 100, or directly inputted by a user through an input unit (described later). Then, the body fat or body sugar sensor may be mounted in the user device 100 in a detachable or detachable form.

In addition, for example, the body fat or body sugar data can be measured by external body fat or body sugar measuring device 130 and transmitted to user device 110. When the user measures a body fat using the body fat or body sugar measuring device 130, the user device 110 can receive the body fat or body sugar data according to the measurement result from the body fat or body sugar measuring device 130. Then, the user device 110 and the body fat or body sugar measuring device 130 may be connected to each other by using short-distance wireless communication (for example, ZigBee, WiFi-Direct, Bluetooth or the like) or the remote wired/wireless communication and transmit and/or receive data. Alternatively, data reception may be performed via a microphone input port of the user device 110. In some cases, the body fat or body sugar data may be directly transmitted from the body fat or body sugar measuring device 130 to the external server 120 without a relay of the user device 110.

The external server 120 can analyze the body fat or body sugar data received from the user device 110 and determine an application execution information according to the analysis result. In addition, the external server 120 can levelize the body fat or body sugar data.

For example, a percentage of the body fat may be levelized via at least two or more steps such as 20 steps of level 1 to 20 or 10 steps of 1 to 10 for 11% to 30% of body fat.

The external server 120 can acquire a percentage of current body fat or a level of body sugar, a degree of change relative to a percentage of previous body fat or a level of body sugar, a percentage of target body fat or a level of body sugar, etc. by analyzing the body fat or the body sugar data, and determine an application execution information.

For example, the application execution information includes information on the body fat or body sugar, or character information and/or game information differentiated in accordance with the result of measurement of the body fat or body sugar, as information necessary for execution of the body fat or body sugar related application of the user device 110. The character information may relate to attributes such as a shape, a type, a facial expression, action, size, sound of the character or combinations thereof according to a measurement information of the body fat or the body sugar or a measurement result the body fat or body sugar. In addition, the game information may include game operation information including items related to game operation, weapons, a life, or levels, which are related to or not related to the character. The application execution information may include an image related to increase/decrease of a body fat to be displayed during a game execution, when the body fat or body sugar related application includes a game application.

The external server 120 can control the user device 110 to execute the body fat or body sugar related application corresponding to the determined application execution information, by transmitting the determined application execution information to the user device 110. For example, when the application execution information includes a measurement information of body fat or body sugar, the external server 120 may control the user device 110 to display at least one of a level of current body fat or body sugar, a change history of body fat or body sugar, and a level of target body fat or body sugar, as an execution of the body fat or body sugar related application.

When the body fat or body sugar related application includes a game application and the application execution information includes a differentiated character information according to the measurement result of body fat or body sugar, the external server 120 may control the user device 110 to execute the game application by selecting the character image and the character level according to the character information.

In the exemplary embodiment of the present invention, When the body fat or body sugar related application includes a game application and the application execution information includes a differentiated game information according to the measurement result of body fat or body sugar, the external server 120 may control the user device 110 to execute the game application by selecting at least one of an accessible game level and a target game level according to the game information.

The user device 110 may execute the body fat or body sugar related application corresponding to the application execution information received from the external server 120 and output the result of execution thereof. This will be described later.

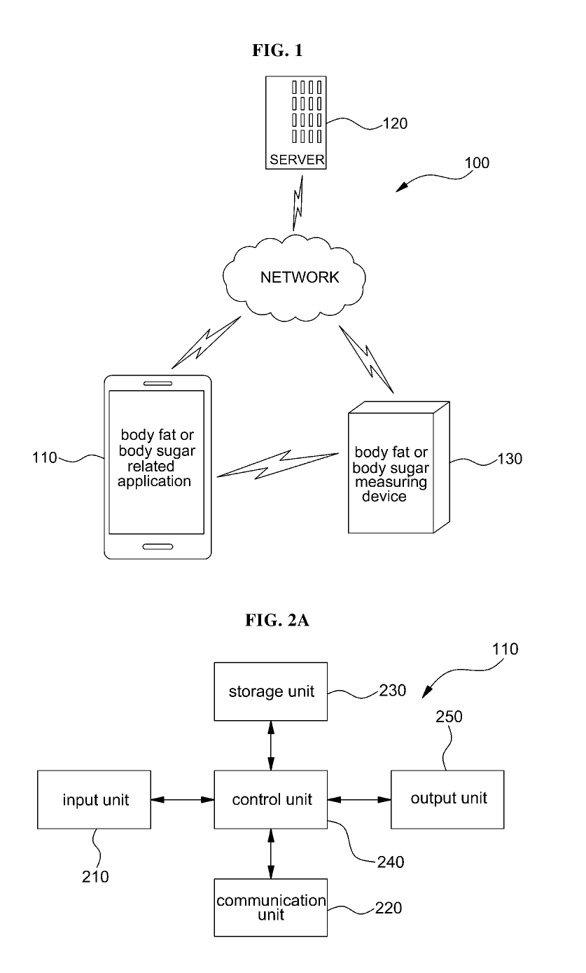

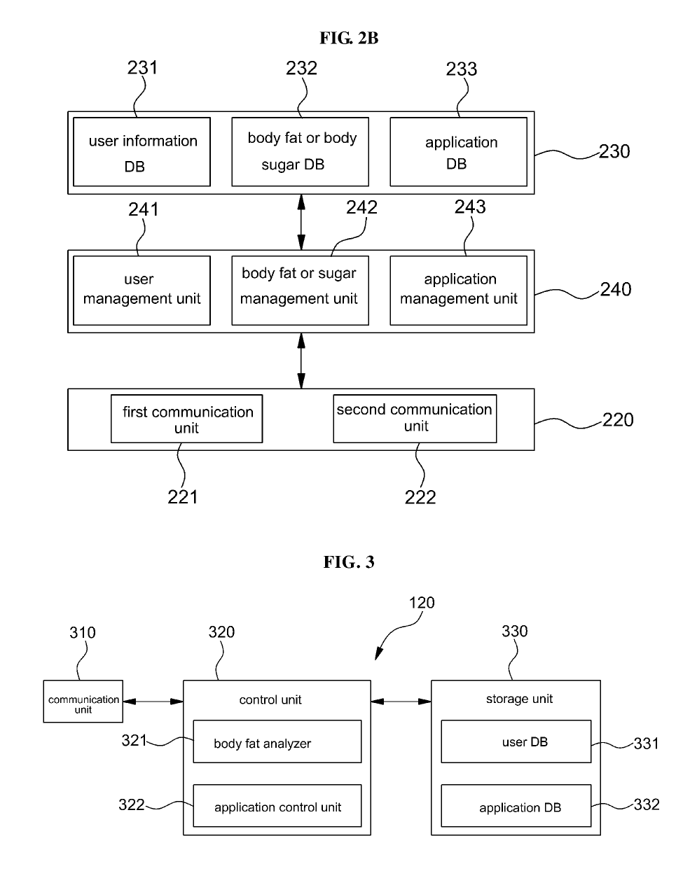

FIG. 2A illustrates a block diagram of a user device having a function managing a body fat or body sugar according to an embodiment of the present invention and FIG. 2B illustrates a detailed configuration of a storage unit/control unit/communication unit shown in FIG. 2A.

Referring to FIG. 2A, the user device 110 includes an input unit 210 for receiving user information, a communication unit 210 for transmitting the body fat or body sugar data to an external server and receiving the application execution information according to body fat or body sugar data from the external server 120, a storage unit 230 for storing at least one body fat or body sugar related application, a control unit 240 for executing the body fat or body sugar related application based on the application execution information, and an output unit 230 for outputting execution results of the body fat or body sugar related application.

Referring to 2A, the user device 110 may receive the user information from a user through the input unit 210. Further, the user device 110 may receive the user information in the execution state (or activation state) of the body fat or body sugar related application. Then, the body fat or body sugar related application may provide an input screen for the user information and induce the user to input the user information.

For example, the input unit 210 may include a keypad, a button, a touch screen, a voice input means, an operation input means or a combination thereof, but the present invention is not limited thereto and the input unit 210 can include any other input means if the other input means comprise an element having a function of information input from the user. The user information may include information related to the user (especially, human body information) such as sex, age, weight, height, amount of muscle, a level of body water, and the like. Deletion/modification of the user information including the user information.

Referring to FIG. 2B, the user management unit 241 controls the user information database (DB) 231 to store the user information, and controls the first communication unit 221 to transmit the user information to the external server 120. When receiving a deletion/addition/change of the user information from the user through the input unit 210, the user management unit 241 can control the user information DB 231 and the first communication unit 221 to apply the deletion/addition/change of the user information to the user information DB 231.

In some cases, the user information may be obtained from an external device (for example, a body measuring device) via the communication unit 220. Then, the input unit 210 can receive a user information acquisition instruction from the external device.

The user device 110 acquires the body fat or body sugar data under a control of the control unit 240 and transmits the acquired body fat or body sugar data to the external server 120 through the communication unit 220.

FIG. 2B, the second communication unit 222 is a module for communicating with the external body fat or a body sugar measuring device 130, and receives the body fat or body sugar data under the control of the body fat or body sugar management unit 242. As described above, the body fat or body sugar data may also be obtained through detecting using the body fat or body sugar sensor mounted on the user device 110 or direct input of the user.

Further, the body fat or body sugar management unit 242 may control the body fat or body sugar database (DB) 232 to store the obtained body fat or body sugar data, and control the first communication unit 221 to transmit the obtained body fat or body sugar data to the external server 120. The body fat or body sugar DB 232 may store the body fat or body sugar data for a predetermined period under a control of the body fat or body sugar management unit 242 and may update a change history of the body fat or body sugar based on the obtained body fat or body sugar data. The first communication unit 221 transmits all of the obtained body fat or body sugar data under a control of the body fat or body sugar management unit 242 or extracts and transmits only an information necessary for determining an application execution information among the obtained body fat or body sugar data.

The user device 110 may receive the application execution information according to the body fat or body sugar data from the external server 120 through the communication unit 220 under a control of the control unit 240. Further, the output unit 250 may output a notification sound, a notification vibration, a notification message, and the like in order to notify a reception on the application execution information, when the application execution information is received, under a control of the control unit 240.

Here, the application execution information according to the body fat or body sugar data may be different or differentiated according to the result of measurement of body fat or body sugar based on the body fat or body sugar data, as information necessary for execution of the body fat or body sugar related application. For example, the application execution information may include a measurement information of body fat or body sugar, a character information, or a game information.

FIG. 2B, the application management unit 243 may control the first communication unit 221 to receive the application execution information from the external server 120, and may control the application database (DB) 233 to store the received application execution information associated to a corresponding body fat or body sugar related application. Not only the application DB 233 may store a plurality of applications including the body fat or a body sugar related application, but also the application DB 233 store the application execution information such that a corresponding body fat or body sugar related application are identified, under a control of the application management unit 243.

The user device 110 may execute the corresponding body fat or body sugar related application corresponding to the received application execution information and output the result of the execution through the output unit 250, under a control of the control unit 240. Furthermore, when the application execution information is received, the control unit 240 may automatically execute the corresponding body fat or body sugar related application, or the control unit 240 may execute the corresponding body fat or body sugar related application in response to the execution command which is input from the user. In addition, if the body fat or body sugar related application is already being executed, the control unit 240 may induce the user to select whether to execute the application corresponding to the application execution information or automatically switch to an execution state corresponding to the application execution information.

Here, the output unit 250 includes a display unit (e.g., LCD, LED, OLED, touch screen, etc.), an audio output unit (e.g., a speaker), a vibration output unit, and does not limit its name or form if it is a means of performing the output function.

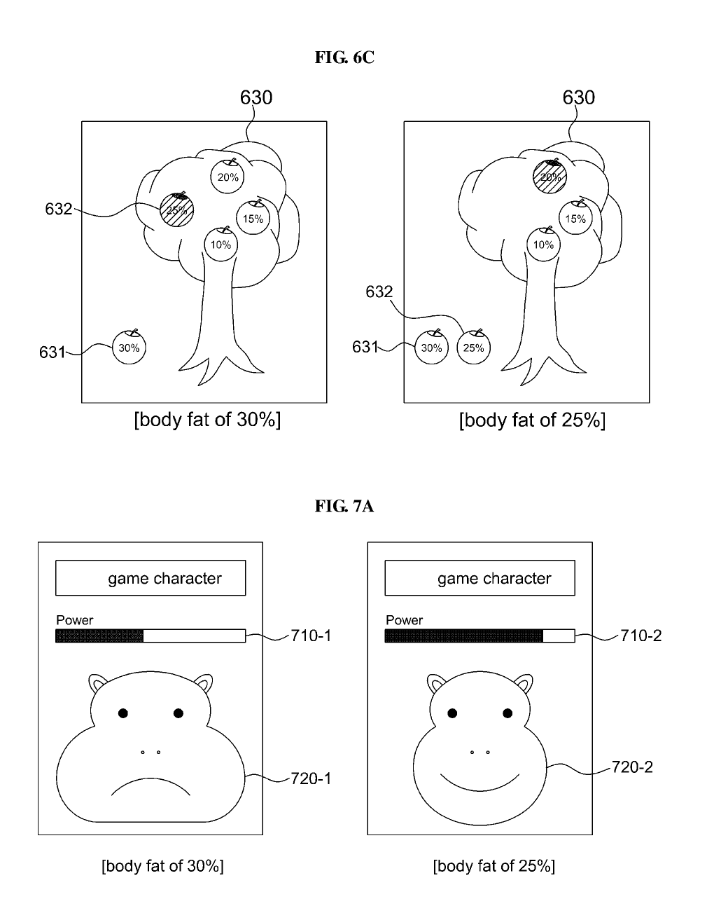

For example, when the application execution information includes the measurement information of body fat or body sugar, the control unit 240 may execute the body fat or body sugar related application so as to display at least one of a level of current body fat or body sugar, a change history of the body fat or body sugar and the target body fat, and output the execution result of the body fat or body sugar related application through the output unit 250. This will be described later referring to FIGS. 6A to 6C.

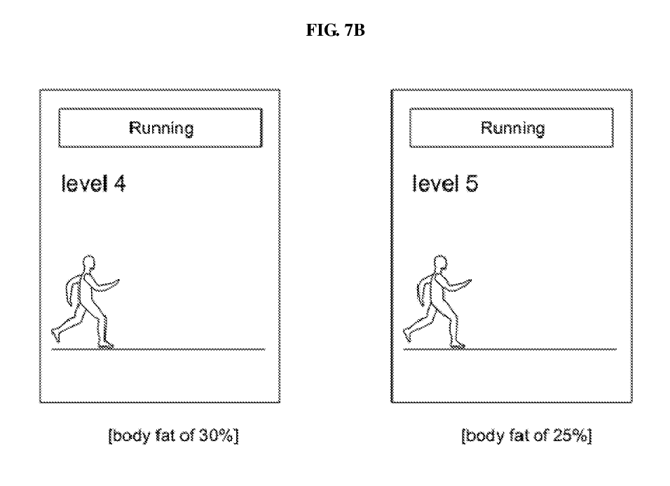

In addition, when the body fat or body sugar related application includes a game application and the application execution information includes a differentiated character information corresponding to a measurement result of the body fat or body sugar, the controller 240 may execute the game application by selecting at least one of a character image and a character level according to the differentiated character information and output the execution result of the body fat or body sugar related application through the output unit 250. This will be described later referring to FIG. 7A.

When the body fat or body sugar related application includes a game application and the application execution information includes a differentiated game information corresponding to a measurement result of the body fat or body sugar, the controller 240 may execute the game application by selecting at least one of an accessible game level and a target game level according to the differentiated game information and output the execution result of the body fat or body sugar related application through the output unit 250. This will be described later referring to FIG. 7B. In another embodiment, the game information may include at least one of an item, a life, and weapon related to a game. For example, with respect to game operation, when the level of body fat is high, in the game application, instant food such as pizza, hamburger and cola as an item can be defined as an obstacle to be avoided, and foods to be helped to decrease a body fat such as vegetables and fruits can be defined as life or an item to be collected. These game levels or elements in the game application such as items, life, and weapons may be operated in conjunction with the information of user's body fat, thereby encouraging the user to improve an improper eating habits associated with an obesity or adult diseases.

In one embodiment, the character operating in the game application may perform various operations for game implementation such as running, jumping, swimming, and flying in the game, and the operations of the character may be interworked with the information of body fat of the user.

For example, operations such as jumping, swimming, and flying of the character may be quantified as an amount of calories to be actually consumed, and the user is advised to do an exercise corresponding to the quantified amount of calories, or the user may be required to input on a quantitative value of an amount of calories which the user may calculate based on a type, a time and a distance of exercises after performing actually the exercises, to be reflected in the progress of the game application.

In one embodiment, the control unit 240 may share information on a target game level with at least one another user via a social network service (SNS). For example, when the user achieves the target game level, the control unit 240 not only uploads the achieved game level on the social network service, but also be provided a game level achieved by another user or a ranking information of game level through the social network service. This may be possible by accessing the social network service through the game application, or by independently accessing the social network service separately from the game application. In addition, users who share information on the target game level through the social network service may provide comments on the game level of anther users as well as themselves.

Information related to body fat through social network services can be integrated with additional information related to health care, encouragement, or suggestion by implementing a ranking or competition mode with anther friends who have achieved the goal.

Furthermore, the control unit 240 can control the output unit 250 to display an image (e.g., a game item icon) related to increase/decrease of body fat during an execution of the game application, and when acquiring the image related to increase/decrease of body fat by the game character, it is possible to adjust a power and/or image of the game character or to adjust an accessible game level.

In FIG. 2B, the configuration of each of the communication unit 220, the storage unit 230, and the control unit 240 is subdivided according to the corresponding function and/or role. However, this is only an embodiment, and therefore, do not exclude doing the corresponding function and/or role in an integrated configuration.

FIG. 3 illustrates a block diagram of a server having a function managing a body fat or body sugar according to an embodiment of the present invention

Referring to FIG. 3, the server 120 includes a communication unit 310 for receiving the body fat or body sugar data from the user device 110 and transmitting the application execution information corresponding to the received body fat or body sugar data to the user device 110, a control unit 320 for analyzing the body fat or body sugar data, determining the application execution information according to the analysis result, and controlling the user device 110 to execute the body fat or body sugar related application according to the determined application execution information, and a storage unit 330 for storing at least one of data and application execution information.

The server 120 may receive the body fat or body sugar data from the user device 110 through the communication unit 310 under a control of the control unit 320. In some cases, the communication unit 310 may directly receive the body fat or body sugar data from the body fat or body sugar measuring device 130 without a relay of the user device 110. This is the case, for example, when a power of the user device is turned off, or a network condition of between the user device 110 and the server 120 or the user device 110 and the body fat or body sugar measurement device is poor, the communication unit 310 may directly receive the body fat or body sugar data from the body fat or body sugar measuring device 130.

The server 120 may analyze the received body fat or body sugar data under a control of the controller 320 and determine the application execution information according to the analysis result.

More specifically, the body fat analyzer 321 may analyze the body fat or body sugar data using a user information stored in the user database (DB) 331. The user DB 331 may store user's body fat information (for example, the body fat or body sugar data, a target body fat, a change history of body fat, and a level of previous body fat, and a reference body fat information, etc.).

For example, the body fat analyzer 321 may determine whether the user's body fat is increased or decreased by comparing a level of current body fat obtained from the body fat or body sugar data with a level of pre-stored previous body fat. The body fat analyzing unit 321 may determine the target body fat based on the current body fat obtained from the body fat or the body sugar data, according to at least one of a weight a height, an age, and a sex of the pre-stored user. The body fat analyzing unit 321 may determine a level of an insufficiency or an excess of the body fat information obtained from the body fat or the body sugar data based on the reference body fat.

The application control unit 322 may determine the application execution information by utilizing the application database (DB) 332 in accordance with a result of the body fat analysis. The application DB 332 includes at least one application provided or can be provided to the user device 110 by the server 120 and an information related thereto, for example, the application DB 332 may store an information (Hereinafter, referring to as application execution information) necessary for executing the body fat or body sugar related application provided to the user device 110. For example, the application execution information may include an information related to application download and installation, an address of application provider server, and various information provided when an application is executed, and the like. Particularly, in the case of a body fat or body sugar related application, the application execution information may include a measurement information of body fat or body sugar indicating a level of body fat or body sugar and in the case of a game application, the application execution information may include a character information or a game information corresponding to a level of body fat or body sugar.

For example, the application control unit 322 may determine, as the application execution information, an image or a level corresponding to 25% of body fat when it is determined that the current body fat is 25%, as a result of analyzing the body fat or body sugar data, or determine an image representing a level of change relative to the previous body fat, when it is determined that the current body fat relative to the previous body fat is decreased or increased, as a result of analyzing the body fat or body sugar data, or determine an image representing the target body fat, when the target body fat is determined based on the current body fat. A plurality of images or levels mapped to a plurality of body fat percentages may be stored in the application DB 332 as application execution information.

For example, the application control unit 322 may determine, as the application execution information, a game character or a level of game character corresponding to 25% of body fat when it is determined that the current body fat is 25%, as a result of analyzing the body fat or body sugar data, or determine an accessible game level or a target game level corresponding to 25% of body fat, when it is determined that the current body fat is 25%, as a result of analyzing the body fat or body sugar data. A plurality of game character images, levels of game characters, and target game levels mapped to a plurality of body fat percentages may be stored in the application DB 332 as application execution information.

The server 120 may control the user device 110 to execute the body fat or body sugar related application in accordance with the determined application execution information under a control of the controller 320. This means that, substantially, the server 120 can control the application execution operation performed on the user device 110, since the application execution information is provided to the user device 110 by the server 120.

For example, the server 120 may transmit an application execution information and a signal (hereinafter, a command signal) for instructing an application execution according to the application execution information, to the user device 110, through the communication unit 310, the user device 110 may identify the command signal, thereby executing the corresponding body fat or body sugar related application based on the application execution information corresponding the identified command signal.

Although the control unit 320 and the storage unit 330 are classified as each configuration according to a corresponding function/role, it is only one embodiment. Therefore, it does not exclude that the function/role is performed by one integrated component.

FIG. 4 is a flowchart for illustrating a method of managing a body fat or body sugar according to an embodiment of the present invention, and the present invention will be described only in the case where the body fat or body sugar data is obtained from an external body fat or body sugar measuring device 130. For an effective description of the present invention, the system elements shown in FIG. 1 are referenced.

Referring to FIG. 4, when the user device 110 is connected to the body fat or body sugar measuring device 130 so as to enable data communication between the user device 110 and the body fat or body sugar measuring device 130 (S411), the user device 110 executes the body fat or body sugar related application (S412). More specifically, the user device 110 executes the body fat or body sugar related application when an execution command is input from a user, even if the user device 110 is connected to the body fat or body sugar measuring device 130. In another example, the user device 110 may automatically execute the body fat or body sugar related applications while detecting a connection between the user device 110 and the body fat or body sugar measuring device 130. Of course, even if the user device 110 is not connected to the body fat or body sugar measuring device 130, the user device 110 may execute the body fat or body sugar related application in response to receiving an execution command from the user.

The user device 110 receives user information in the execution state of the body fat or body sugar related application (S413). For example, the user information may include at least one of gender, age, height, and weight, and may be directly inputted from the user or received from an external device.

The body fat or body sugar measuring device 130 detects the user's body signal and measures the body fat or body sugar data of the user in step S414 and transmits the measured body fat or body sugar data to the user device 110 in step S415. The body fat or body sugar measuring device 130 may transmit the measured body fat or body sugar data after waiting until the connection between the body fat or body sugar measuring device 130 and the user device 110 are recovered or the power of the user device 110 is turned on, when the connection between the body fat or body sugar measuring device 130 and the user device 110 are poor or when the power of the user device 110 is turned off.

The user device 110 transmits the body fat or body sugar data received from the body fat or body sugar measuring device 130 to the server 120 (S416). Alternatively, the body fat or body sugar measuring device 130 may directly transmit the body fat or body sugar data to the server 120 without a relay of the user device 110, when the connection between the user device 110 and the body fat or body sugar measuring device 130 or the user device 110 and the server 120.

The server 120 analyzes the body fat or body sugar data received from the user device 110 (S417), and determines an application execution information according to the analysis result (S418). The server 120 may utilize the user information, the body fat information, or the application related information, pre-stored in the database to analyze the body fat or body sugar data and to determine the application execution information.

The server 120 transmits the determined application execution information to the user device 110 (S419). At this time, the server 120 may add an identification information of the body fat or body sugar related application and an execution command signal to the application execution information and transmit the application execution information including the identification information and the execution command signal.

The user device 110 executes the corresponding body fat or body sugar related application in accordance with the application execution information received from the server 120 in step S420 and outputs the execution result of the body fat or body sugar related application in step S421.

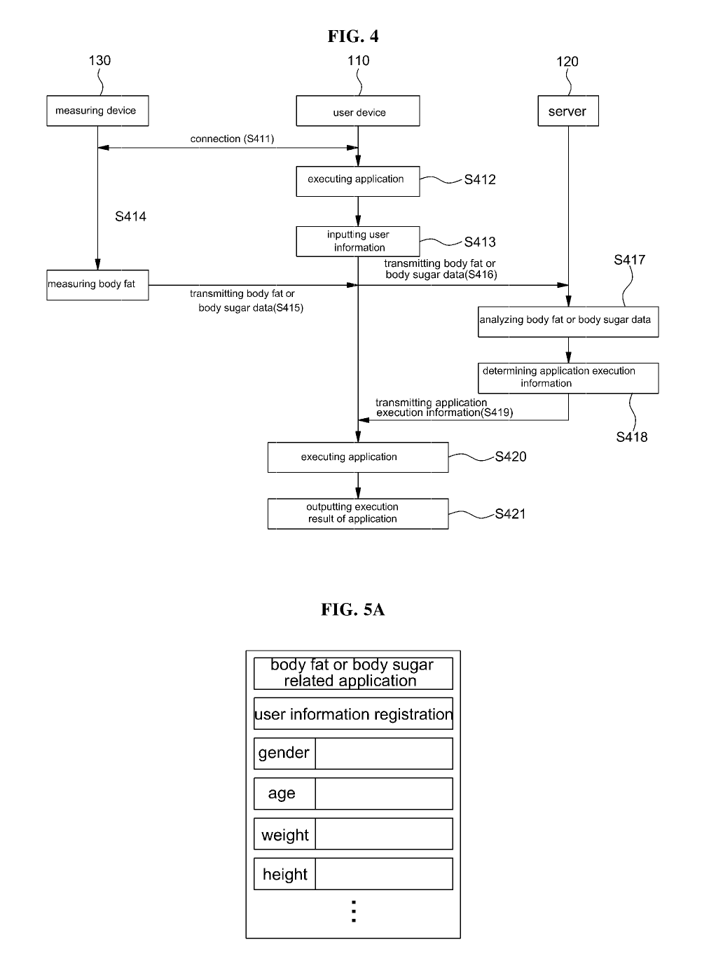

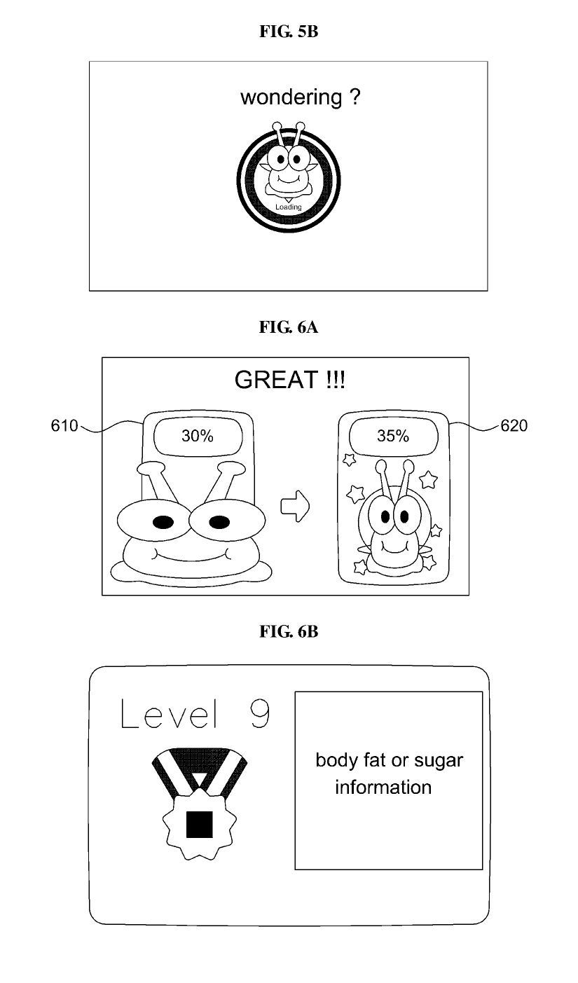

For example, when the user device 110 receives the application execution information and the identification information of the body fat or body sugar related application, the user device 110 identifies the body fat or body sugar related application using the identification information and executes the body fat or body sugar related application using the application execution information. Further, if the user device 110 is currently not executing the body fat or body sugar related application, the user may be notified of the reception of the application execution information and the user device 110 induce the user to input an execution command of the body fat or body sugar related application. Further, if the user device 110 is currently in the execution state of the body fat or body sugar related application, it can be automatically switched to the execution state corresponding to the application execution information according to the user's selection. The output of the execution result of the body fat or body sugar related application will be described in more detail below referencing FIGS. 5A to 7B.

FIGS. 5A and 5B illustrate a screen for a user information registration and a screen for a body fat or body sugar measurement according to an embodiment of the present invention, respectively.

According to FIG. 5A, the user device may display a screen for user information registration, that can receive user information from the user in the execution state of the body fat or body sugar related application. For example, the user information may include gender, age, weight, and height. But the present invention is not limited thereto.

Although it is not shown in the FIG. 5A, the body fat or body sugar related application may provide an edit screen for the user information and allow the user to input a delete/add/change of the user information. For example, the user information registration may be required at the first execution of the body fat or body sugar related application, and editing such as the delete/add/change of the user information may not require a separate time point if the body fat or body sugar related application is in the execution state.

FIG. 5B, the user device displays an image indicating that the body fat or body sugar data is being measured while measuring body fat or body sugar data, and further displays an image informing the measurement progress status. For example, if the image is a specific person/animal image, the user device may display a facial expression to wonder about the measurement result or display the facial expression and/or facial color to change according to the measurement progress status. Alternatively, the user device can display the measurement progress status using the progress bar, display an image indicating the completion of the measurement upon completion of measurement, or display a measurement completion message.

FIGS. 6A to 6C illustrate a screen for displaying measurement results of body fat or body sugar according to an embodiment of the present invention.

FIG. 6A, when the application execution information is an image representing the previous and current body fat percentage, the user device displays an image 610 representing the previous body fat percentage and an image 620 representing the current body fat percentage, as the execution result of the body fat or body sugar related application, thereby intuitively providing a change history of the body fat to the user. Then, the images 610 and 620 may be different in size/color/shape/facial expression according to the corresponding body fat percentage, and phrases (for example, ‘Great’ while decreasing the body fat percentage, ‘Bad’ while increasing the body fat percentage) corresponding to decrease/increase of the body fat percentage, may be displayed together.

According to FIG. 6B, when the application execution information is an image representing the current body fat percentage, the user device can display a level indicating the current fat percentage, as a result of execution of the body fat or body sugar related application. Since the level indicating the body fat percentage may belong to a different level according to the body fat percentage, the level representing the current body fat percentage is different between the case where the body fat percentage is 25% and the case where the body fat percentage is 30%. For example, assuming that levels 1 to 20 are set for the percent body fat of 11% to 30%, the level 15 can be displayed for the current body fat percentage of 25%.

Further, as information on the current body fat percentage, it is possible to further provide a recommended exercise information, a recommended diet information, a reference body fat percentage, and the like.

Referring to FIG. 6C, when the application execution information is an image representing the target body fat percentage, the user device can display an image representing the achieved body fat percentage and the target body fat percentage, as a result of execution of the body fat or body sugar related application. For example, the body fat percentage can be displayed by using the fruit 630 which is opened on the tree.

More specifically, since the existing target body fat percentage was 30% and the current body fat percentage is 30%, the target body fat percentage has been achieved, so that the fruit 631 corresponding to the achieved target body fat percentage 30% falls on the floor and the fruit 632 corresponding to the next target body fat percentage is displayed to be identified. Then, when the body fat percentage of 25% is achieved, the fruits 631, 632 corresponding to the target body fat percentages of 25% and 30% fall on the floor, and the fruit 633 corresponding to the next target body fat percentage of 20% is displayed to be identified. Thus, the user can intuitively confirm a change history of his or her target body fat and a next target body fat percentage. In addition, the target body fat percentage may be set according to the user's selection or according to a determination of the application control unit 322, and in particular, in the case of user selection, the body fat percentage corresponding to the fruit selected by the user can be set as the target body fat percentage.

Although it is not shown in the drawing, the user device can display the target body fat percentage in more various ways, for example, by displaying an image acquired, by jumping, the fruit corresponding to the target body fat percentage from a tree having a plurality of fruits per target body fat percentage, or the image of the fruit corresponding to the target body fat percentage is placed on the tree growing, as the current body fat percentage becomes closer to the target body fat percentage.

FIG. 7A illustrate a screen for displaying a game character information differentiated according to measurement results of a body fat or body sugar, and FIG. 7B a screen for displaying a game information differentiated according to measurement results of a body fat or body sugar. Hereinafter, the case where the body fat or the body sugar related application includes a game application will be described.

FIG. 7A, when the application execution information is the current body fat percentage, the character image corresponding to the current body fat percentage, and the character ability (or a level or a power of the character), the user device displays the character image corresponding to the current body fat percentage, as the execution result of the game application, and can authorize the character to use the character power corresponding to the current body fat percentage.

More specifically, if the current body fat percentage is 30%, the user device sets the game character to have the power 710-1 corresponding to the body fat percentage of 30%, and displays the character image 720-1 corresponding to the body fat percentage of 30%. Alternatively, if the current body fat percentage is 25%, the user device sets the game character to have the power 710-2 corresponding to the body fat percentage of 25%, and displays the character image 720-2 corresponding to the body fat percentage of 25%. For example, the lower the body fat percentage, the higher the character power, and the character image can be set to be slim or to make a smiling face.

Although it is not shown in the drawings, it is possible to provide a character image list differentiated according to the current body fat percentage (for example, the lower the body fat percentage, the more selectable images or the higher power images can be selected), and to induce the user to select a character image.

Referring to FIG. 7B, when the application execution information includes the current body fat percentage and the accessible game level corresponding to the current body fat percentage, the user device may provide up to accessible game levels corresponding to the current body fat percentage.

More specifically, assuming that the game level is 1 to 10 and the accessible game level is 10 to 1 for the body fat percentage of 1 to 50%, it is possible to access game levels 1 to 4 when the current body fat percentage is 30%, and %, it is possible to access game levels 1 to 5 when the current body fat percentage is 25%. The lower the body fat percentage, the higher the level of accessible game can be set. In addition, if it is possible to set a target game level by reflecting the target body fat percentage, for example, when the current body fat percentage is 30%, the target game level can be set to level 5 by setting the target body fat percentage to 25%, and when the current body fat percentage is 25%, the target game level can be set to level 6, by setting the target body fat percentage to 20%.

Further, the user device may display an image related to increase or decrease of the body fat during a game execution. Therefore, the user can always be aware of the increase or decrease of body fat during the execution of the game application.

For example, a user device, as an image related to body fat increases, junk food icons to the body, such as high-calorie and fast food can be displayed, and when the game character obtains the junk food icons to the body, a low score is given to the game character, or the power or energy of the game character is reduced. In addition, the user device, as an image related to body fat decreases, a vegetable or good food icons to the body, such as low-calorie can be displayed, and when the game character obtains the good food icons to the body, the power or energy of the game character is increased, or the level of accessible game is up, or the game character is allowed to have special abilities.

Although it is not shown in the drawing, when the game application includes an exercise assisted game application that induces a user to jog, run, walk, etc., the higher the body fat percentage, the higher game level corresponding to exercise intensity can be accessed.

Also, although it is not shown in the drawings, by providing ranking information of the game application, as application execution information, to the user device, the user device can allow the user to identify users' body fat percentage and their given position through comparison between the user and other users.

Further, according to another embodiment of the present invention, some or all of the application execution information may be determined at the user device. In this case, the information necessary for determining the application execution information is pre-stored in the user device, or can be acquired by being connected to the server when necessary. In addition, it may be seen that the same or similar operation is performed in the user device by citing the description of an analysis of the body fat or body sugar data and a determination of application execution information in the server.

The above-described embodiments of the present invention may be implemented as a method, system, or system for producing software, firmware, hardware, products or combinations thereof, such as, for example, a cloud computing environment, using programming and/or storage media, and the article of manufacture may comprise a computer program, a carrier wave, or a media.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of managing body fat or body sugar using a user device, the method comprising:
transmitting a body fat or body sugar data to an external server;
receiving application execution information according to the body fat or body sugar data from the external server;
executing a body fat or body sugar related application based on the application execution information; and
outputting an execution result of the body fat or body sugar related application,
wherein the application execution information is based on a result of levelizing the body fat or body sugar data.

2. The method of claim 1, the step of executing a body fat or body sugar related application, comprising:
executing the body fat or body sugar related application so as to display at least one of a level of current body fat or body sugar, a change history of body fat or body sugar and target level of body fat or body sugar, when the application execution information includes a measurement information of body fat or body sugar.

3. The method of claim 1, the step of executing a body fat or body sugar related application, comprising:
the body fat or body sugar related application comprises a gaming application,
executing the gaming application by selecting at least one of a character image and a character level according to a differentiated character information, when the application execution information includes the differentiated character information corresponding to the measurement result of body fat or body sugar.

4. The method of claim 1, the step of executing a body fat or body sugar related application, comprising:
the body fat or body sugar related application comprises a gaming application,
executing the gaming application by selecting at least one of an accessible game level and a target game level according to a differentiated game information, when the application execution information includes the differentiated game information corresponding to the measurement result of body fat or body sugar.

5. The method of claim 4, further comprising:
sharing an information about the target game level with at least one user via a social network service (SNS).

6. The method of claim 4, wherein the step of outputting comprises:
displaying an image related to decrease or increase of the body fat or the body sugar, when the game application is executed according to the application execution information.

7. A method of managing body fat or body sugar using a server, the method comprising:
receiving a body fat or body sugar data from a user device;
analyzing the body fat or body sugar data and determining application execution information based on the analysis result;
transmitting the determined application execution information to the user device; and
controlling the user device to execute a body fat or body sugar related application in accordance with the determined application execution information,
wherein the application execution information is based on a result of levelizing the body fat or body sugar data.

8. A method of managing body fat or body sugar using a system, the method comprising:
transmitting, by a user device, a body fat or body sugar data to a server;
analyzing, by the server, the body fat or body sugar data received from the user device and determining application execution information according to the analysis result;
transmitting, by the server, the determined application execution information to the user device; and
executing, by the user device, a body fat or body sugar related application corresponding to the application execution information received from the server,
wherein the application execution information is based on a result of levelizing the body fat or body sugar data.

9. A user device having a function of managing body fat or body sugar, the user device comprising:
a storage unit for storing a body fat or body sugar related applications;
a communication unit for transmitting body fat or body sugar data to an external server and receiving application execution information according to the body fat or body sugar data from the external server;
a control unit for executing the body fat or body sugar related application based on the application execution information; and
an output unit for outputting a result of execution of the body fat or body sugar related application,
wherein the control unit controls operations of at least one of the storage unit, the communication unit, and the output unit, and
wherein the application execution information is based on a result of levelizing the body fat or body sugar data.

10. The user device of claim 9, further comprising:
an input unit for receiving user information including at least one of a user's age, a user's weight, a user's height, and a user's gender.

11. The user device of claim 9,
wherein the body fat or body sugar data are obtained by at least one of receiving from an external body fat or body sugar measuring device, detecting through a body fat or body sugar sensor included in the user device, and inputting directly from a user.

12. The user device of claim 9,
wherein the control unit executes the body fat or body sugar related application, so as to display at least one of a level of current body fat or body sugar, a change history of body fat or body sugar and a target level of body fat or body sugar, when the application execution information includes a measurement information of body fat or body sugar.

13. The user device of claim 9,
wherein the control unit executes a game application by selecting at least one of a character image and a character level according to a differentiated character information, when the body fat or body sugar related application comprises the gaming application and the application execution information includes the differentiated character information corresponding to the measurement result of body fat or body sugar.

14. The user device of claim 9,
wherein the control unit executes a game application by selecting at least one of an accessible game level and a target game level according to a differentiated game information, when the body fat or body sugar related application comprises the gaming application and the application execution information includes the differentiated game information corresponding to the measurement result of body fat or body sugar.

15. The user device of claim 9,
wherein the user device includes at least one of a mobile device and a stationary device.

16. A server having a function of managing body fat or body sugar, the server comprising:
a communication unit for receiving a body fat or body sugar data from a user device and transmitting application execution information corresponding to the body fat or body sugar data to the user device;
a controller for analyzing the body fat or body sugar data, determining the application execution information according to the analysis result, and controlling the user device to execute a body fat or body sugar related application according to the determined application execution information; and
a storage unit for storing at least one of the body fat or body sugar data and the application execution information;
wherein the control unit controls operations of at least one of the communication unit and the storage unit, and
wherein the application execution information is based on a result of levelizing the body fat or body sugar data.

17. The server of claim 16,
wherein the control unit controls the user device to display at least one of a level of current body fat or body sugar, a change history of body fat or body sugar and target level of body fat or body sugar, when the application execution information includes a measurement information of body fat or body sugar.

18. The server of claim 16,
wherein the control unit controls the user device to select at least one of a character image and a character level according to a differentiated character information, when the body fat or body sugar related application comprises the gaming application and the application execution information includes the differentiated character information corresponding to the measurement result of body fat or body sugar.

19. The server of claim 16,
wherein the control unit controls the user device to select at least one of an accessible game level and a target game level according to a differentiated game information, when the body fat or body sugar related application comprises the gaming application and the application execution information includes the differentiated game information corresponding to the measurement result of body fat or body sugar.

20. A system having a function of managing body fat or body sugar, the system comprising:
a user device for transmitting a body fat or body sugar data; and
a server for analyzing the body fat or body sugar data received from the user device and determining application execution information according to the analysis result,
wherein the user device executes a body fat or body sugar-related application corresponding to the application execution information received from the server,
wherein the application execution information is based on a result of levelizing the body fat or body sugar data.

21. The system of claim 20,
wherein the user device executes the body fat or body sugar related application, so as to display at least one of a level of current body fat or body sugar, a change history of body fat or body sugar and target level of body fat or body sugar, when the application execution information includes a measurement information of body fat or body sugar.

22. The system of claim 20,
wherein the user device executes a game application by selecting at least one of a character image and a character level according to a differentiated character information, when the body fat or body sugar related application comprises the gaming application and the application execution information includes the differentiated character information corresponding to the measurement result of body fat or body sugar.

23. The system of claim 20,
wherein the user device executes a game application by selecting at least one of an accessible game level and a target game level according to a differentiated game information, when the body fat or body sugar related application comprises the gaming application and the application execution information includes the differentiated game information corresponding to the measurement result of body fat or body sugar.
